(19) United States
(12) Patent Application Publication
(10) Pub. No.: US 2021/0293667 A1
(43) Pub. Date: Sep. 23, 2021

(54) PARTICLE OBTAINING METHOD, PARTICLE CAPTURING CHAMBER, AND PARTICLE ANALYSIS SYSTEM

(71) Applicant: SONY CORPORATION, TOKYO (JP)

(72) Inventors: TASUKU YOTORIYAMA, TOKYO (JP); KENSUKE KOJIMA, TOKYO (JP); YOSHIAKI KATO, TOKYO (JP); SHIN MASUHARA, TOKYO (JP)

(21) Appl. No.: 17/250,579

(22) PCT Filed: May 23, 2019
(86) PCT No.: PCT/JP2019/020375

§ 371 Date: Feb. 4, 2021

(30) Foreign Application Priority Data
Aug. 15, 2018 (JP) 2018-152936

(51) Int. Cl. G01N 1/14; B01L 3/00; G01N 15/10

(52) U.S. Cl. CPC G01N 1/14; B01L 3/502761; G01N 15/10; G01N 2001/1025; B01L 2300/0829; B01L 2200/0689; B01L 2300/123; B01L 2200/0647

(57) ABSTRACT

To provide a technology for obtaining a desired particle. The present technology provides a particle obtaining method including a particle capturing step of capturing a particle in a well, a sealing step of sealing the well with a sheet, and a particle obtaining step of obtaining the particle from a selected well after the sealing step. Furthermore, the present technology also provides a particle capturing chamber provided with a particle capturing unit including at least one well for capturing a particle therein, and a sealing unit including a sheet for sealing the well, in which a distance between the well and the sheet is adjustable.

### TECHNICAL FIELD

The present technology relates to a particle obtaining method, a particle capturing chamber, and a particle analysis system. More specifically, this relates to a particle obtaining method, a particle capturing chamber, and a particle analysis system used for analyzing one particle.

### BACKGROUND ART

Attention is focused on a single cell analysis technology. In the single cell analysis technology, cells are captured one by one in a large number of microwells arranged on a plane, respectively, and morphology of each cell is individually observed to analyze a characteristic of each cell and/or a reaction of each cell with reagent is analyzed using, for example, fluorescence as an index.

Several technologies for performing the single cell analysis have been proposed so far. For example, Patent Document 1 described below discloses a method for capturing and analyzing a cell set, the method provided with capturing the cell set containing a cell subgroup with a pore set of a substrate; delivering a reagent amount to the pore set through a manifold fluid-communicated with the pore set; guiding a cell migration tool into one pore of the pore set that contains one cell of the cell subgroup; and receiving the cell of the cell subgroup from the pore to the cell migration tool (claim 1).

Furthermore, there also are several commercially available devices for performing the single cell analysis. As such device, for example, PREP system (Celsee, Inc.), Cell Picking System (AS ONE Corporation.), Amnis™ Imaging Flow Cytometer (Merck KGaA), C1 (Fluidigm Corporation), Chromium (10× Genomics) and the like are available.

### CITATION LIST

### Patent Document

Patent Document 1: US Patent Application Publication No. 2017/0073745 Specification

### SUMMARY OF THE INVENTION

### Problems to be Solved by the Invention

There is a case where it is required to take out only one desired cell after performing a single cell analysis. An object of most of the devices mentioned above is cell analysis or analysis of gene contained in the cell, and it is not possible to recover the desired cell. Furthermore, although some devices have a function for taking out only one cell, if there is a more efficient cell recovery technology, this is considered to be more beneficial for recovering one cell.

An object of the present technology is to provide a technology for recovering one desired cell.

### Solutions to Problems

The present inventors have found that the above-described problem may be solved by a specific particle capturing method or a specific particle capturing chamber.

That is, the present technology provides a particle obtaining method provided with a particle capturing step of capturing a particle in a well, a sealing step of sealing the well with a sheet, and a particle obtaining step of obtaining the particle from a selected well after the sealing step.

At the particle capturing step, the particle may be captured in the well by suction to a side opposite to a settling side of the particle through a pore provided in the well.

At the sealing step, the well may be sealed with the sheet by adjustment of a distance between the well and the sheet.

The particle obtaining method may further be provided with a selecting step of observing the particle in the well and selecting the particle that should be obtained after the sealing step.

The particle obtaining method may further be provided with a hole making step of making a hole in a portion that seals the selected well of the sheet after the sealing step, in which the particle may be obtained from the hole made at the hole making step at the particle obtaining step.

Furthermore, the present technology also provides a particle capturing chamber provided with a particle capturing unit that includes at least one well for capturing a particle in the well, and a sealing unit including a sheet for sealing the well, in which a distance between the well and the seat is adjustable.

The distance between the well and the sheet may be adjusted by movement of the sheet toward the well or movement of the well toward the sheet, and the well may be sealed with the sheet by the movement.

The sheet may be transparent.

The sheet may further include an adhesive layer.

The sheet may include a piezoelectric body, and the piezoelectric body may emit an elastic wave.

The sheet may further include a reagent layer.

According to one aspect of the present technology, the sealing unit may further include a support layer stacked on the sheet, the sheet may be peeled from the support layer by a pressure generated by injection of fluid, and the distance between the well and the sheet may be adjusted.

The fluid may be at least one selected from water, air, oil, and a cell culture solution.

According to another aspect of the present technology, a pore used for capturing the particle in the well by suction is provided on each of the at least one well, and a pore sealing unit including a sheet for sealing the pore may further be provided.

According to still another aspect of the present technology, the particle capturing unit may include an elastic material, the well may be moved toward the sheet by a pressure generated by injection of fluid, and the distance between the well and the sheet may be adjusted.

A pore used for capturing the particle in each well by suction is provided on each of the at least one well, and the well may open toward a settling side of the particle.

Furthermore, the present technology also provides a particle analysis system provided with a particle capturing chamber provided with a particle capturing unit including at least one well for capturing a particle in the well and a sealing unit including a sheet for sealing the well, an analysis unit that analyzes the captured particle, and a light source that perforates the sheet on the basis of information of the particle analyzed by the analysis unit.

With the present technology, desired cells may be obtained one by one. For example, according to the present technology, only the cell having a certain characteristic may be selectively recovered out of a plurality of cells.

Note that the effect of the present technology is not necessarily limited to the effects herein described and may be any of the effects described in the present specification.

### BRIEF DESCRIPTION OF DRAWINGS

FIG. 1A is a schematic diagram of an example of a particle capturing chamber used for performing a particle obtaining method of the present technology.

FIG. 1B is a schematic diagram of an example of the particle capturing chamber used for performing the particle obtaining method of the present technology.

FIG. 1C is an example of a flow chart of the particle obtaining method of the present technology.

FIG. 2A is a view illustrating an example of a state in which a well is sealed with a sheet.

FIG. 2B is a view illustrating an example of a state in which a particle in the sealed well is observed.

FIG. 2C is a view illustrating an example of a state in which the particle is recovered from the sheet on which a hole is made.

FIG. 3 is a schematic diagram of an example of a particle capturing chamber used for performing a particle obtaining method of the present technology.

FIG. 4 is a view for explaining steps included in the particle obtaining method of the present technology.

FIG. 5A is a schematic diagram of an example of a particle capturing chamber used for performing a particle obtaining method of the present technology.

FIG. 5B is a view illustrating a manufacturing example of the particle capturing chamber.

FIG. 6 is a view for explaining steps included in the particle obtaining method of the present technology.

FIG. 7 is a schematic diagram of an example of a particle capturing chamber used for performing a particle obtaining method of the present technology.

FIG. 8 is a view for explaining steps included in the particle obtaining method of the present technology.

FIG. 9 is a schematic diagram of an example of a particle capturing chamber used for performing a particle obtaining method of the present technology.

FIG. 10 is a view for explaining steps included in the particle obtaining method of the present technology.

FIG. 11 is a schematic diagram illustrating an example of a particle analysis system of the present technology.

### MODE FOR CARRYING OUT THE INVENTION

Hereinafter, a preferred mode for carrying out the present technology is described. Note that embodiments hereinafter described are representative embodiments of the present technology, and the scope of the present technology is not narrowed by them. Note that the present technology is described in the following order.

1. First embodiment (particle obtaining method)

(1) Description of first embodiment

(1-1) Particle capturing chamber

(1-2) Particle obtaining method

(2) First example of first embodiment (particle obtaining method)

(2-1) Example of particle capturing chamber

(2-2) Example of operation procedure

(2-3) Example of operation procedure

(3) Second example of first embodiment (particle obtaining method)

(3-1) Example of particle capturing chamber

(3-2) Example of operation procedure

(3-3) Example of particle capturing chamber

(3-4) Example of operation procedure

(4) Third example of first embodiment (particle obtaining method)

(4-1) Example of particle capturing chamber

(4-2) Example of operation procedure

2. Second embodiment (particle capturing chamber)

3. Third embodiment (particle analysis system)

### 1. First Embodiment (Particle Obtaining Method)

### (1) Description of First Embodiment

A particle obtaining method of the present technology includes a particle capturing step of capturing a particle in a well, a sealing step of sealing the well with a sheet, and a particle obtaining step of obtaining the particle from a selected well after the sealing step. By sealing the well in which the particle is captured with the sheet, it is possible to maintain a state in which one particle is isolated in one well. Then, by making a hole only in a portion that seals the well containing a desired particle, for example, it is possible to selectively obtain only the desired particle. That is, the present technology enables positive selection of the particle.

Hereinafter, first, an example of a tool used for performing the particle obtaining method of the present technology is described, and next, the steps included in the particle obtaining method of the present technology are described.

(1-1) Particle Capturing Chamber

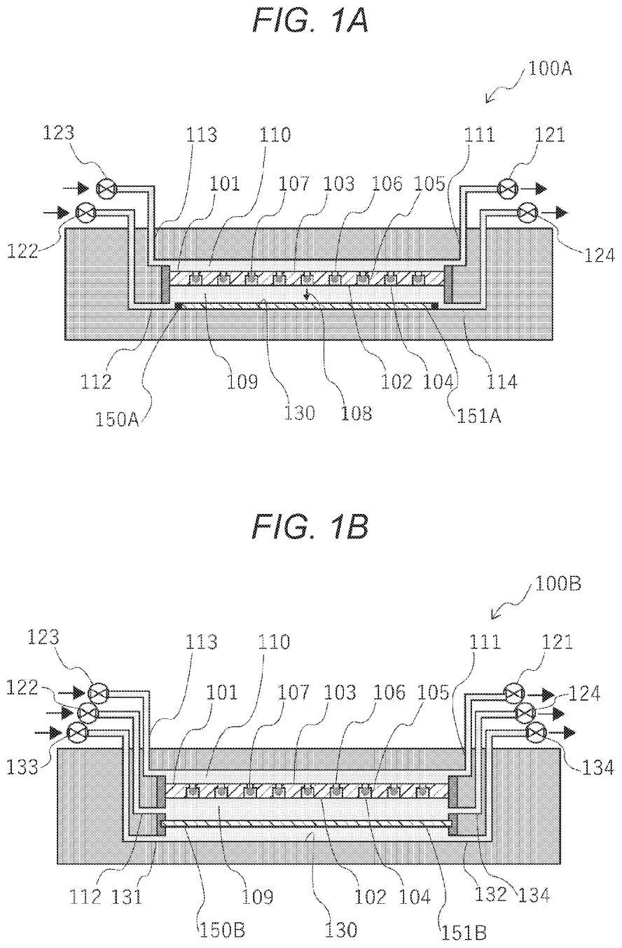

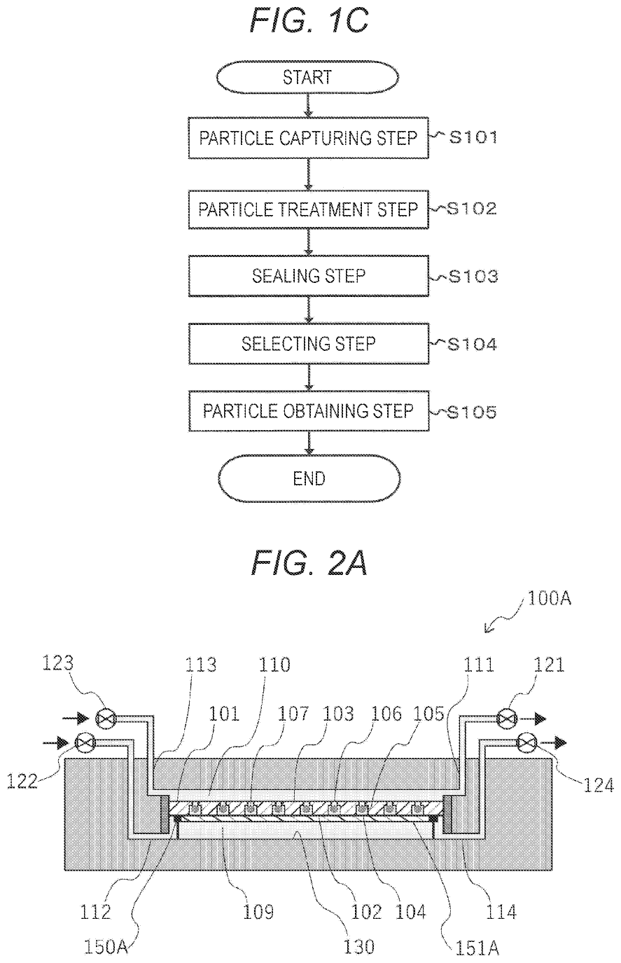

FIG. 1A is a schematic diagram of an example of a particle capturing chamber used for performing the particle obtaining method of the present technology. A particle capturing chamber 100A illustrated in FIG. 1A is provided with a particle capturing unit 101. The particle capturing unit 101 includes a particle capturing surface 102 and a surface 103 facing an opposite side. A plurality of wells 104 is provided on the particle capturing surface 102. A pore 106 is provided on a bottom 105 of each of the wells. The pore 106 penetrates from the bottom 105 of the well to the surface 103 on the side opposite to the particle capturing surface 102. The well 104 has such a dimension that a particle 107 may be accommodated therein. The pore 106 has such a dimension that the particle 107 does not pass therethrough. The pore 106 is used for capturing the particle in the well 104 by suction.

Note that FIG. 1A is a schematic diagram illustrating a state in which the particle 107 is captured in the well 104, and it is not required that the particle 107 be in the well 104 before a particle capturing process starts.

The particle capturing unit 101 is arranged in the particle capturing chamber 100A so as to divide a space in the particle capturing chamber 100A into upper and lower two spaces. The particle capturing chamber 100A is arranged so that gravity acts on the particle 107 in a direction of arrow 108. That is, the particle 107 settles in the direction of arrow 108. Then, out of the two spaces separated by the particle capturing unit 101, the lower space is referred to as a particle settling side space 109, and the upper space is referred to as a space 110 on an opposite side of the settling side space.

The particle capturing unit 101 (especially an area in which the well 104 is formed) may be formed by using, for example, a material commonly used in the technical field regarding a micro channel. Examples of the material may include, for example, glass such as borosilicate glass and quartz glass, for example; plastic resins such as an acrylic resin, a cycloolefin polymer, and polystyrene, for example; rubber materials; and silicone resins such as PDMS, for example. For example, by using the silicone resin such as PDMS as the material for forming the particle capturing unit 101, sealing with a sheet 151 described below may be made more reliable.

The particle capturing chamber 100A is provided with a particle capturing channel unit 111, a first fluid supply channel unit 112, a second fluid supply channel unit 113, and a fluid discharge channel unit 114. The particle capturing channel unit 111 and the second fluid supply channel unit 113 are connected to the opposite side space 110. The first fluid supply channel unit 112 and the fluid discharge channel unit 114 are connected to the settling side space 109.

The particle capturing channel unit 111, the first fluid supply channel unit 112, the second fluid supply channel unit 113, and the fluid discharge channel unit 114 are provided with valves 121, 122, 123, and 124, respectively.

The particle capturing chamber 100A further includes a sealing unit 150A. The sealing unit 150A includes a sheet 151A stacked on a bottom surface 130 in the settling side space 109. The sheet 151A is for sealing the well 104. The sealing unit 150A is configured to be able to adjust a distance between the sheet 151A and the well 104. By eliminating the distance, that is, by bringing the well 104 into contact with the sheet 151A, the well 104 is sealed with the sheet 151A.

According to one aspect of the present technology, the particle capturing chamber 100A may be configured to be able to move the sheet 151A toward the particle capturing unit 101. That is, the particle capturing chamber 100A may be configured to be able to adjust the distance between the sheet 151A and the well 104 while keeping a distance between the bottom surface 130 and the well 104 fixed. The movement brings the particle capturing unit 101 and the sheet 151A into contact with each other, and the well 104 is sealed with the sheet 151A. For example, the well 104 may be sealed with the sheet 151A by rising of the sealing unit 150A rises, for example.

According to another aspect of the present technology, the particle capturing chamber 100A may be configured such that the particle capturing unit 101 may move toward the bottom surface 130, or that the bottom surface 130 may move toward the particle capturing unit 101. Since the particle capturing chamber 100A is configured in this manner, the distance between the well 104 and the sheet 151A may be adjusted. The particle capturing unit 101 and the sheet 151A come into contact with each other by the movement, so that the well 104 is sealed with the sheet 151A.

An example of more specific configuration and operation of the sealing unit 150A is described in “(2) First example of first embodiment (particle obtaining method)”, “(3) Second example of first embodiment (particle obtaining method)”, and “(4) Third example of first embodiment (particle obtaining method)” described below.

The sheet 151A is preferably transparent. For example, the sheet 151A may have such transparency that the particle in the well may be observed through the sheet 151A. Therefore, at an observing step described below, the particle in the well sealed with the sheet 151A may be observed.

The sheet 151A may preferably be formed by using a material capable of being perforated by irradiation with light such as a laser beam, for example. The light may be infrared light, for example, especially near-infrared light, and the sheet 151A may be a sheet containing or coated with an infrared light absorber, for example, especially a near-infrared light (NIR) absorber. Examples of the near-infrared light absorber may include indocyanine green, phthalocyanine, porphyrin, CNT, or a metal nanoparticle, for example. Preferably, the near-infrared light absorber is an inorganic material. For example, CuO and P₂O₅ may be contained in or applied to the sheet 151A as the near-infrared light absorber. By using the inorganic material, it is possible to prevent generation of active oxygen due to near-infrared light irradiation, and it is possible to prevent damage to the particle (especially a cell).

As a more specific example of the material of the sheet 151A, for example, there may be a resin sheet (especially a silicone resin sheet) containing the near-infrared light absorber or a glass sheet, and a resin sheet (especially a silicone resin sheet) coated with the near-infrared light absorber or a glass sheet.

According to one aspect of the present technology, the sheet 151A preferably includes an adhesive layer. The adhesive layer may be provided, for example, on a surface in contact with the particle capturing unit 101. The adhesive layer adheres to the particle capturing unit 101 (especially the particle capturing surface 102), so that the well is more reliably sealed. As a material that forms the adhesive layer, preferably a pressure-sensitive adhesive, more preferably an acrylic pressure-sensitive adhesive, and still more preferably an acrylic pressure-sensitive adhesive exhibiting tackiness even in water may be used. As such pressure-sensitive adhesive, a commercially available material may be used, or a well-known material (for example, that disclosed in Japanese Patent Application Laid-Open No. 2002-97428) may be used.

According to another aspect of the present technology, the sheet 151A may include a piezoelectric body, and the piezoelectric body may emit an elastic wave. More specifically, the sheet 151A may be a piezoelectric plate and may emit an elastic wave. The piezoelectric body makes it possible to disperse the particles or convey the particle to a desired position. Furthermore, in a case where the sheet 151A and the particle capturing unit 101 come into contact with each other, the elastic wave is emitted from the piezoelectric body, and the contact may be detected by the piezoelectric body. Then, in response to the detection of the contact, the adjustment of the distance between the sheet 151A and the well 104 may be stopped.

According to still another aspect of the present technology, the sheet 151A may include a reagent layer. The reagent layer may be provided on a surface in contact with the particle capturing unit 101 out of two surfaces of the sheet 151A.

The reagent layer may be, for example, a layer containing fluorescent dye as a reagent. The fluorescent dye may emit fluorescence, for example, by coming closer to or coming into contact with a compound that the particle has (especially a compound that the particle has on its surface). The fluorescent dye may be appropriately selected by those skilled in the art.

Alternatively, the reagent layer may be a layer containing a compound that causes emission of fluorescence by the fluorescent dye, that is, it is not required that the compound itself emits fluorescence. The compound may cause the emission of fluorescence from the fluorescent dye, for example, when the fluorescent dye with which the particle that should be detected is labeled comes closer to or comes into contact with the compound. The compound may be appropriately selected by those skilled in the art.

In the particle capturing chamber 100A illustrated in FIG. 1A, the sheet 151A is stacked on the bottom surface 130 in the settling side space 109, but it is not required that the sheet 151A be stacked on the bottom surface 130 in the settling side space 109. FIG. 1B illustrates an example of a particle capturing chamber in which a well sealing sheet is not stacked on the bottom surface of the chamber.

A particle capturing chamber 100B illustrated in FIG. 1B is different from the particle capturing chamber 100A illustrated in FIG. 1A in that a sheet 151B is arranged away from the bottom surface 130 (that is, arranged in the air in the settling side space 109) in place of the sheet 151A stacked on the bottom surface 130 and that channel units 131 and 132 are added. Other components are as described with reference to FIG. 1A. The particle capturing chamber 100B is configured such that a distance between the well 104 and the sheet 151B is adjustable. Especially the particle capturing chamber 100B is configured such that the distance between the well 104 and the sheet 151B may be eliminated, that is, the well 104 and the sheet 151B may come into contact with each other. The well 104 and the sheet 151B come into contact with each other, so that the well 104 is sealed with the sheet 151A. In this manner, in the present technology, the well sealing sheet included in the sealing unit may be arranged away from the bottom surface 130 of the settling side space 109.

According to one aspect of the present technology, the particle capturing chamber 100B may be configured such that the sheet 151B may move toward the particle capturing unit 101, or that the particle capturing unit 101 may move toward the sheet 151B. Since the particle capturing chamber 100B is configured in this manner, the distance between the well 104 and the sheet 151B may be adjusted. The particle capturing unit 101 and the sheet 151B come into contact with each other by the adjustment, so that the well 104 is sealed with the sheet 151B. For example, by introducing fluid from the channel units 131 and 132 to pressurize, the sheet 151B may move toward the particle capturing unit 101.

All of the contents described regarding the sheet 151A (for example, the transparency, material, adhesive layer, piezoelectric body, reagent layer and the like) also apply to the sheet 151B.

In the present technology, the particles are required to be captured one by one, for example. The particles may include, for example, biological microparticles such as cells, microorganisms, living body-derived solid components, and liposomes, synthetic particles such as latex particles, gel particles, and industrial particles and the like, but there is no limitation. The cells may include animal cells and plant cells. Examples of the animal cells may include tumor cells and blood cells, for example. The microorganisms may include bacteria such as Escherichia coli, fungi such as yeast and the like. Examples of the living body-derived solid components may include solid crystals generated in the living body, for example. The synthetic particles may be particles including, for example, organic or inorganic polymer materials, metal or the like. The organic polymer materials may include polystyrene, styrene-divinylbenzene, polymethyl methacrylate and the like. The inorganic polymer materials may include glass, silica, a magnetic material and the like. The metal may include gold colloid, aluminum and the like. Furthermore, in the present technology, the particle may be a bound substance of a plurality (e.g., two or three) of particles.

In the present technology, the fluid includes liquid and gas. Preferably, the fluid is the liquid. A type of the liquid may be appropriately selected by those skilled in the art depending on a type of the particle. In a case where the particle is, for example, a cell, for example, water, an aqueous solution (for example, a buffer), or a culture solution may be used as the liquid.

In the present technology, the well 104 may open to the particle settling side. That is, an opening of the well 104 may face the particle settling side. Therefore, the particle is captured in the well by suction to the opposite side of the particle settling side.

In the present technology, the well may be configured to open to the opposite side space 110. That is, the particle capturing unit 101 may be arranged such that the particle capturing surface 102 of the particle capturing unit 101 illustrated in FIGS. 1A and 1B faces the opposite side space 110 and the opposite side surface 103 faces the settling side space 109. In this case, the well sealing sheet may be arranged on the ceiling of the opposite side space 110, or the well sealing sheet may be arranged in the air of the opposite side space 109. For example, after the particle enters the well by the settling or suction, the well may be sealed with the well sealing sheet arranged on the ceiling or in the air.

Furthermore, in the present technology, the particle capturing surface 102 may be arranged perpendicular to the direction in which the gravity acts as illustrated in FIGS. 1A and 1B, or may be arranged so as to incline (that is, arranged so as to form an angle other than a right angle with respect to the direction in which the gravity acts). The inclination may be formed by arranging the particle capturing chamber according to the present technology with inclination.

In the present technology, each of the wells may have a shape capable of capturing one particle. For example, an entrance of the well may be, for example, a circle, an ellipse, a polygon, for example, a triangle, a quadrangle (for example, a rectangle, a square, a parallelogram, a rhombuses and the like), a pentagon, a hexagon and the like. In the present technology, the entrance of the well is intended to mean the opening of the well on the surface on which the well is provided of the particle capturing unit. The shape of the entrance of the well may be designed, for example, such that the particle that should be captured may enter the well but the particle that should not be captured cannot enter the well.

In the present technology, the wells 104 may be regularly arranged on the particle capturing surface 102. Regular well arrangement makes it easier to specify a position of the well in which a target particle is captured. As a result, for example, it becomes possible to more easily take out and/or observe the particle captured by the well. For example, the wells may be arranged on the particle capturing surface in a row or a plurality of rows at a predetermined interval, or the wells may be arranged on the particle capturing surface in a grid pattern at a predetermined interval. The interval may be appropriately selected by those skilled in the art depending on, for example, the number of particles to be applied, the number of particles that should be captured and the like. The interval may be, for example, 20 μm to 300 μm, preferably 30 μm to 250 μm, more preferably 40 μm to 200 μm, and still more preferably 50 μm to 150 μm. For example, in a case where the wells are arranged in a grid pattern, the wells may be arranged at the intervals illustrated above in X and Y directions on the particle capturing surface.

In order to manufacture the particle capturing unit 101 (especially the portion in which the well is formed), for example, a 3D optical shaping method using an optical shaping printer or a high-definition 3D printer, an optical shaping method by PDMS resin molding, a method of directly processing glass by a laser, or a method of processing a SiO₂ membrane by a semiconductor process may be used. A device for carrying out these methods may be appropriately selected by those skilled in the art. For example, as a device used for the 3D optical shaping method, there may be, for example, an ACCULAS™ series optical shaping printer. A resin used for the 3D optical shaping may be appropriately selected by those skilled in the art. The resin is, for example, a photocurable resin composition containing one or two or more selected from an acrylic oligomer, an acrylic monomer, an epoxy oligomer, and an epoxy monomer, and may be, for example, an ultraviolet curable resin composition. The resin composition may be cured using the optical shaping printer to form the particle capturing unit 101. By these methods, the particle capturing unit 101 including the well 104 having a desired shape and the pore 106 may be manufactured.

A material of other parts of the particle capturing chamber 100 (especially a material forming a wall surface defining the space in the chamber 100 and that forming a wall surface of the channel connected to the space in the chamber 100) may be appropriately selected by those of skilled in the art. For example, in a case where the particle is the cell, a material not toxic to cell is preferable as the material. Furthermore, in a case of performing fluorescence observation of the captured particle, it is preferable to use a material that does not emit autofluorescence beyond a permissible range. Furthermore, it is preferable to use a material that enables the observation of the particle captured in the well. For observing the particle, for example, at least a part of the chamber, especially the bottom of the settling side space of the chamber may be formed by using a transparent material.

As a material of other parts of the particle capturing chamber 100, for example, a material commonly used in the technical field of the micro channel may be used. Examples of the material may include, for example, glass such as borosilicate glass or quartz glass, for example; plastic resins such as an acrylic resin, a cycloolefin polymer, and polystyrene, for example; or rubber materials such as PDMS, for example. In a case where the particle capturing chamber of the present technology is formed by using a plurality of members, the plurality of members may be formed by using the same material or different materials. Preferably, the bottom surface of the particle capturing chamber 100 (that is, the bottom surface of the settling side space 109) is formed by using a transparent material. Therefore, the particle in the well 104 may be observed through the bottom surface.

(1-2) Particle Obtaining Method

The particle obtaining method of the present technology using the particle capturing chamber 100A illustrated in FIG. 1A is hereinafter described with reference to a flow chart illustrated in FIG. 1C. FIG. 1C is an example of the flow chart of the particle obtaining method of the present technology.

(1-2-1) Particle Capturing Step

At step S101, the particle capturing step of capturing the particle in the well is performed. An example of the particle capturing step using the particle capturing chamber 100A illustrated in FIG. 1A is described below.

A container (not illustrated) for storing fluid containing particles is connected to the first fluid supply channel unit 112. By driving a pump (not illustrated) provided on the first fluid supply channel unit 112, the fluid containing the particles is supplied from the container through the first fluid supply channel unit 112 into the settling side space 109 of the particle capturing chamber 100A.

A pump (not illustrated) is connected to the particle capturing channel unit 111. By driving the pump, the fluid in the particle capturing chamber 100A is sucked out of the opposite side space 110 of the particle capturing chamber 100 through the particle capturing channel unit 111.

Particle capturing by the particle capturing chamber 100A may be performed, for example, by simultaneously performing the supply of the particle-containing fluid from the first fluid supply channel unit 112 and the suction of the fluid from the particle capturing channel unit 111. That is, the particle enters the particle capturing chamber 100A from the first fluid supply channel unit 112, then rises in the settling side space 109. The particle further rises in the settling side space 109 and enters the well 104. The particle rises in the well 104, then comes into contact with an entrance of the pore 106. The pore 106 has such a dimension that the particle cannot pass therethrough, so that the particle is captured in the well 104. In this manner, at the particle capturing step, the suction is performed to the side opposite to the settling side of the particle through the pore 106 provided in the well 104, and the particle is captured in the well 104.

The particle not captured in the well 104 settles to the bottom of the settling side space 109 by the action of the gravity.

By capturing the particle as described above, the particle not captured in the well 104 is suppressed from staying near the well of the particle capturing unit 101, and/or the particle is suppressed from further entering the well in which the particle is already captured. Therefore, in a case where the particle captured in the well 104 is observed, for example, with a microscope arranged below the particle capturing chamber 100A, the particle not captured is located in a position away from the well 104, so that this does not bother the observation.

(1-2-2) Particle Treatment Step

At step S102, a particle treatment step of performing treatment of the particle captured in the well is performed. For example, in a case where the particle is a cell, liquid containing a reagent for stimulating the cell may be supplied from the first fluid supply channel unit 112 or the second fluid supply channel unit 113 into the chamber. The reagent may stimulate the cell. For example, in a case where the particle is the cell, an assay (for example, a biochemical assay) may be performed at the particle treatment step. In the assay, liquid containing a reagent for performing the assay may be supplied from the first fluid supply channel unit 112 or the second fluid supply channel unit 113 into the chamber. Examples of the assay may include an assay for measuring an intracellular calcium ion concentration, an assay for observing an intracellular organelle such as mitochondria, and an assay using a PCR method for observing cell-derived genes, for example.

In the particle obtaining method of the present technology, it is not required that the particle treatment step be performed. Alternatively, in the particle obtaining method of the present technology, the particle treatment step may be performed after the following sealing step or performed as a part of the following selecting step.

(1-2-3) Sealing Step

At step S103, the sealing step of sealing the particle captured in the well is performed. The sealing of the well 104 by the sealing unit 150A may be performed by adjusting the distance between the sheet 151A forming the sealing unit 150A and the well 104.

According to one aspect of the present technology, the sheet 151A and the particle capturing surface 102 may be brought into contact with each other by moving or deforming the sheet 151A toward the well 104 (or the particle capturing surface 102). By this contact, the well 104 is sealed with the sheet 151A.

According to another aspect of the present technology, the sheet 151A and the particle capturing surface 102 may be brought into contact with each other by moving the particle capturing unit 101 (or the particle capturing surface 102) toward the sheet 151A. By this contact, the well 104 is sealed with the sheet 151A.

According to still another aspect of the present technology, the sheet 151A and the particle capturing surface 102 may be brought into contact with each other by moving both the particle capturing unit 101 (or the particle capturing surface 102) and the sheet 151A toward each other. By this contact, the well 104 is sealed with the sheet 151A.

As described above, the sheet 151A and the particle capturing surface 102 come into contact with each other by the adjustment of the distance, so that the well 104 is sealed with the sheet 151A.

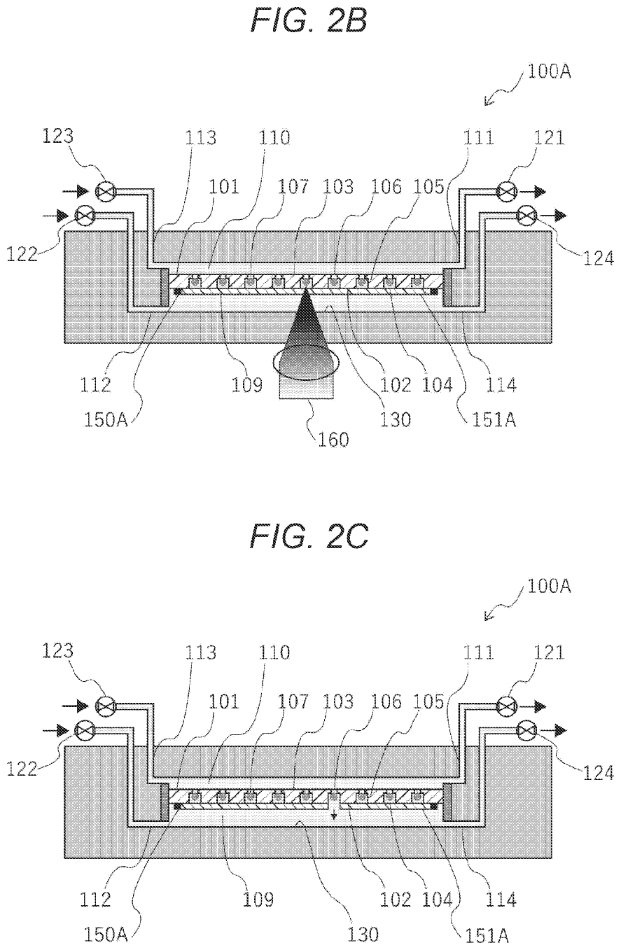

An example of a state in which the well 104 is sealed with the sheet 151A is illustrated in FIG. 2A. In FIG. 2A, the well 104 is sealed with the sheet 151A of the raised sealing unit 150A. By sealing the well 104 with the sheet 151A in this manner, it is possible to prevent the particle from leaving the well 104. Therefore, it is also possible to stop the suction through the particle capturing channel unit 111, for example. This may prevent the particle from being damaged by the suction.

(1-2-4) Selecting Step

At step S104, the selecting step of selecting a particle that should be obtained may be performed. After the well 104 is sealed with the sheet 151A, the particle sealed in the well 104 may be observed. The particle that should be obtained may be selected by the observation. The observation may preferably be performed by an observation device arranged on the particle settling side. The observation may be carried out from below the particle capturing chamber 100, that is, it may be observed from the particle settling side. The observation device may be, for example, an inverted microscope 160 as illustrated in FIG. 2B, and the observation may be performed through an objective lens of the inverted microscope. The observation may be, for example, bright-field observation or fluorescence observation. In these observations, a change in particle over time may be observed.

(1-2-5) Particle Obtaining Step

At step S105, the particle obtaining step of obtaining the particle selected at the selecting step is performed. At the particle obtaining step, a desired particle is obtained from the well 104. In order to obtain the particle from the well 104, a hole is made on the portion that seals the well containing the selected particle of the sheet 151A by, for example, a laser beam, especially an infrared laser beam. In this manner, the particle obtaining step may further include a hole making step of making a hole on the portion that seals the selected well of the sheet after the sealing step. For example, in a case where the sheet 151A contains the near-infrared light absorber described above, the hole is made on the portion of the sheet 151A by irradiating the portion of the sheet 151A with a near-infrared laser beam.

For example, as illustrated in FIG. 2C, after the hole is made, the particle is expelled from the well 104 through the hole. Expulsion of the particle may be performed by, for example, free fall. Alternatively, the particle may be expelled from the well 104 by a differential pressure generated between the settling side space 109 and the opposite side space 110. In order to generate the differential pressure, for example, fluid may be introduced from the particle capturing channel unit 111 to the opposite side space 110, or fluid may be introduced from the second fluid supply channel unit 113 to the opposite side space 110.

The particle expelled from the well 104 is discharged out of the particle capturing chamber 100 by suction by a pump (not illustrated) connected to the fluid discharge channel unit 114, for example, and recovered in a container (not illustrated) connected to the fluid discharge channel unit 114, for example. In this manner, the particle is obtained from the hole made at the hole making step.

As described above, the desired particle is recovered at the particle obtaining step. A plurality of desired particles may be recovered by repeating, for example, the selecting step and the particle obtaining step out of the procedures described above.

### (2) First Example of First Embodiment (Particle Obtaining Method)

According to one aspect of the present technology, the sealing unit may further include a support layer stacked on the sheet. In this aspect, the sheet is peeled from the support layer by a pressure generated by injection of fluid and the distance between the well and the sheet is adjusted.

An example of a particle capturing chamber in this aspect is hereinafter described, and an example of a particle obtaining method using the particle capturing chamber is next described.

(2-1) Example of Particle Capturing Chamber

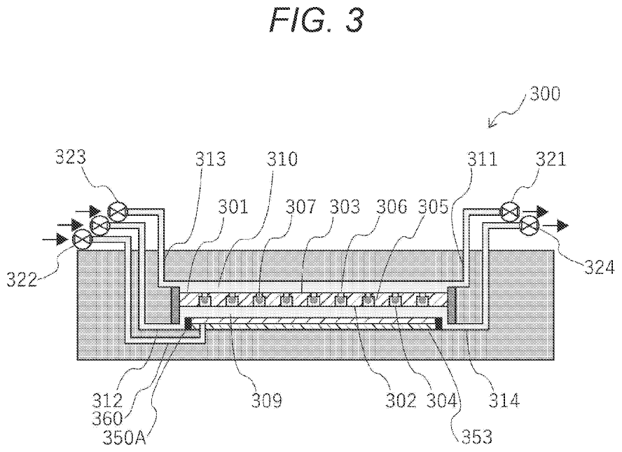

FIG. 3 illustrates an example of a particle capturing chamber of the present technology. A particle capturing chamber 300 illustrated in FIG. 3 is provided with a particle capturing unit 301 as the particle capturing chamber 100A illustrated in FIG. 1 described above. As the particle capturing unit 101 in FIG. 1, the particle capturing unit 301 includes a particle capturing surface 302, a surface 303 on an opposite side, a plurality of wells 304 on the particle capturing surface 302, and a pore 306 penetrating from a bottom of the well 304 to the opposite side surface 303. The particle capturing unit 301 divides the interior of the chamber 300 into a particle settling side space 309 and a space 310 on an opposite side.

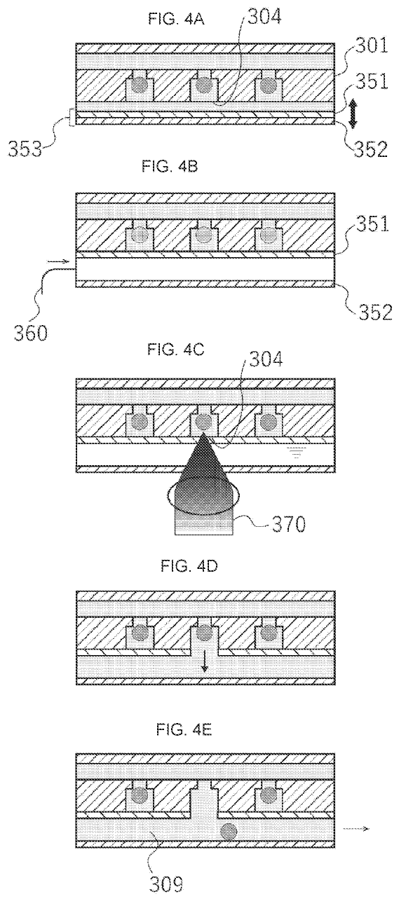

The particle capturing chamber 300 includes a sealing unit 350. The sealing unit 350 includes a stacked body 353 having a two-layered structure. As illustrated in FIG. 4(A), the stacked body 353 includes a well sealing sheet 351 and a support layer 352 on which the well sealing sheet 351 is stacked. These two layers are peelable. The support layer 352 may be a layer different from a bottom surface 330, or may be the bottom surface 330 of the settling side space 309 of the particle capturing chamber 300. A thickness of the well sealing sheet 351 is preferably 3 μm to 50 μm, and more preferably 5 μm to 30 μm.

Furthermore, the particle capturing chamber 300 is provided with a peeling fluid supply channel unit 360 for supplying fluid between the two layers to peel the two layers from each other. The peeling fluid supply channel unit 360 is configured to be able to introduce the fluid between the well sealing sheet 351 and the support layer 352.

The particle capturing chamber 300 is provided with a particle capturing channel unit 311, a first fluid supply channel unit 312, a second fluid supply channel unit 313, and a first fluid discharge channel unit 314. As described above, the particle capturing chamber 300 is also provided with the peeling fluid supply channel unit 360.

As described above, a total of five channel units are connected to the particle capturing chamber 300. A valve and a pump (not illustrated) are provided on each of the above-described five channel units.

The particle capturing channel unit 311 and the second fluid supply channel unit 313 are connected to the opposite side space 310.

The first fluid supply channel unit 312 and the first fluid discharge channel unit 314 are connected to the settling side space 309.

(2-2) Example of Operation Procedure

An example of a particle obtaining method using the particle capturing chamber 300 is hereinafter described. Although a case where a cell is used as a particle is hereinafter described, a particle other than a cell may also be obtained by the particle capturing chamber 300.

(Step a)

A particle capturing step of capturing the particle in the well is performed. At the particle capturing step, in the particle capturing chamber 300, the cell is captured in the well as described in “(1-2-1) Particle capturing step” described above, for example. As a result of the cell capturing, one cell is captured in each well 304 as illustrated in FIG. 4(A).

(Step b)

The cell captured in the well is subjected to treatment with drug. Alternatively, the cell captured in the well is subjected to an assay. The assay may be, for example, a biochemical assay for observing an intracellular calcium ion concentration or an intracellular organelle (for example, mitochondria). It is not necessary that the treatment be performed, that is, step b may be omitted.

(Step c)

A sealing step of sealing the well with the sheet is performed. By adjusting a distance between the stacked body 353 and the well 304, the well 304 is sealed. For example, by moving the stacked body 353 toward the well 304, the stacked body 353 may come into contact with the particle capturing unit 301 to seal the well 304. Alternatively, by moving the particle capturing unit 301 toward the stacked body 353, the particle capturing unit 301 may come into contact with the stacked body 353 to seal the well 304. By the sealing, the cell is sealed in a space defined by the well 304 and the well sealing sheet 351.

After the sealing, a negative pressure applied for capturing the particle may be released. For example, suction through the particle capturing channel unit performed at the particle capturing step may be stopped. This makes it possible to avoid a load or damage on the cell due to the suction.

(Step d)

After step c, fluid such as gas or liquid, for example, is injected from the peeling fluid supply channel unit 360 between the well sealing sheet 351 and the support layer 352 of the stacked body 353. The injected fluid may be at least one (one or a combination of two or more) selected from, for example, water, air, oil, and cell culture solution. Therefore, the support layer 352 is peeled from the well sealing sheet 351 as illustrated in FIG. 4(B).

(Step e)

The fluid that fills a space between the well sealing sheet 351 and the support layer 352 may be replaced with fluid for observation suitable for observation of the cell with a microscope. Preferably, the fluid for observation has a refractive index equivalent to that of the well sealing sheet 351 and/or support layer 352. This makes it easier to observe the cell. The fluid for observation is preferably liquid, and more preferably oil (for example, silicone oil and the like), water, an aqueous solution (for example, a buffer), or a culture solution.

For example, in a case where the peeling is performed by air injection at step d, the air fills the space. In a case where the cell is observed through the space filled with the air, there is a case where it is difficult to observe the cell in the well due to a difference in refractive index between the liquid in the well and the air. Therefore, by exchanging the air with the liquid having the refractive index equivalent to that of the liquid in the well, it is possible to observe the cell more easily.

(Step f)

The cell in the well 304 is observed. For example, it may be observed whether or not the cell has a predetermined characteristic as a result of the treatment or assay performed at step b. In a case where neither the treatment nor assay is performed, it may be observed, for example, whether or not the cell in the well has a predetermined shape. These observations may be performed, for example, by an inverted microscope 370 arranged below the particle capturing chamber 300 as illustrated in FIG. 4(C). A cell that should be obtained is selected by the observation of the cell in the well 304.

(Step g)

A portion corresponding to the well in which the cell selected at step f is captured of the well sealing sheet 351 is burned out by a laser beam. Therefore, a hole is made in the sheet portion that seals the selected well as illustrated in FIG. 4(D). In a case where the well sealing sheet 351 contains an infrared light absorber, an infrared laser beam may be used as the laser beam.

(Step h)

As illustrated in FIG. 4(E), the cell is expelled from the well through the hole made at step g and move into the settling side space 309. The cell expelled from the well may be recovered outside the particle capturing chamber 300 through the fluid discharge channel unit 314, for example. For this recovery, for example, suction by a pump (not illustrated) connected to the fluid discharge channel unit 314 may be performed.

At steps a to h described above, it is possible to selectively recover only a desired cell. Furthermore, a plurality of desired cells may be selectively recovered by repeating steps g and h. Furthermore, by making a plurality of holes at step g, a plurality of selected particles may be collectively recovered at step h.

(2-3) Example of Operation Procedure

Another example of the particle obtaining method using the particle capturing chamber 300 is hereinafter described.

(Step a)

A fluorescent labeling process is performed on the cell subjected to the particle obtaining process by the particle capturing chamber 300. A fluorescent label may be, for example, dye that stains a cell membrane, dye that stains an intracellular organelle, or various biomarkers. One fluorescent label may be used, or a plurality of fluorescent labels may be used.

(Step b)

By using liquid containing the cells subjected to the fluorescent labeling process at step a, cell capturing is performed in the particle capturing chamber 300 as described in “(1-2-1) Particle capturing step” described above.

(Steps c to h) Steps c to h described in “(2-2) Example of operation procedure” described above are performed.

At steps a to h described above, it is possible to selectively recover only a desired cell. Furthermore, a plurality of desired cells may be selectively recovered by repeating steps g and h. Furthermore, by making a plurality of holes at step g, a plurality of selected particles may be collectively recovered at step h.

### (3) Second Example of First Embodiment (Particle Obtaining Method)

According to another aspect of the present technology, the sheet for sealing the well may be deformed from a state of being arranged at a predetermined distance from the well to a state of adhering to the particle capturing unit. Furthermore, it is also possible that a pore used for capturing the particle inside by suction is provided on each of the at least one well, and that a pore sealing unit including a sheet for sealing the pore is further provided.

An example of the particle capturing chamber in this aspect is hereinafter described, and further, an example of a particle obtaining method using the particle capturing chamber is described.

(3-1) Example of Particle Capturing Chamber

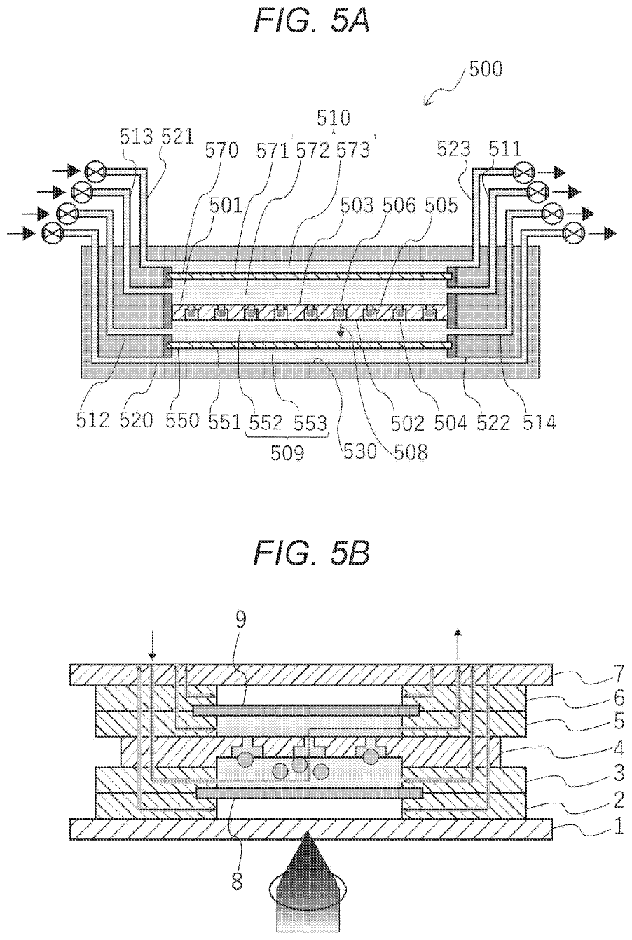

FIG. 5A illustrates an example of a particle capturing chamber of the present technology. A particle capturing chamber 500 illustrated in FIG. 5A is provided with a particle capturing unit 501. The particle capturing unit 501 includes a particle capturing surface 502 and a surface 503 facing an opposite side. The particle capturing surface 502 is provided with a plurality of wells 504. A pore 506 is provided on a bottom 505 of each of the wells. The pore 506 penetrates from the bottom 505 of the well to the surface 503 on the side opposite to the particle capturing surface 502. The particle capturing chamber 500 is arranged such that gravity acts on a particle 507 in a direction of arrow 508. A well 504 has such a dimension that the particle 507 may be accommodated therein. The pore 506 has such a dimension that the particle 507 does not pass therethrough.

The particle capturing unit 501 is arranged in the particle capturing chamber 500 so as to divide a space in the particle capturing chamber 500 into a particle settling side space 509 and a space 510 on an opposite side.

The settling side space 509 is provided with a sealing unit 550. The sealing unit 550 includes a connection end to a well sealing sheet 551 and a chamber inner wall. The settling side space 509 is divided into upper and lower two spaces, that is, a first settling side space 552 and a second settling side space 553 by the well sealing sheet 551 of the sealing unit 550. The well sealing sheet 551 is arranged in parallel with the particle capturing surface 502 of the particle capturing unit 501 at a predetermined distance. That is, the first settling side space 552 is defined by the particle capturing unit 501 and the well sealing sheet 551. The second settling side space 553 is not in contact with the particle capturing unit 501 and is defined by the well sealing sheet 551 and a bottom surface 530 of the chamber 500.

The well sealing sheet 551 is deformable so as to be adherable to the particle capturing surface 502 of the particle capturing unit 501 in a case of sealing the well. That is, the well sealing sheet 551 is deformable from a state of being arranged in parallel with the particle capturing surface 502 at a predetermined distance to a state of adhering to the particle capturing surface 502 of the particle capturing unit 501 as described above. In this deformation, a connection position of the well sealing sheet 551 to the inner wall of the chamber 500 does not have to change. Since the well sealing sheet 551 is deformable in this manner, a distance between the well and the well sealing sheet 551 may be adjusted. The well sealing sheet 551 comes into contact with the particle capturing surface 502 by the adjustment of the distance, so that the well is sealed with the well sealing sheet 551.

The well sealing sheet 551 may be formed by using a material that enables such deformation. The material may be appropriately selected by those skilled in the art, and examples thereof may include polyvinylidene chloride, polyethylene, polypropylene, and polyvinyl chloride, for example. The well sealing sheet 551 may be a stacked body of two or more types of resin layers formed by using any of these materials.

The opposite side space 510 is provided with a pore sealing unit 570. The pore sealing unit 570 includes a connection end to a pore sealing sheet 571 and the chamber inner wall. The opposite side space 510 is divided into a first opposite side space 572 and a second opposite side space 573 by the pore sealing sheet 571 of the pore sealing unit 570. The pore sealing sheet 571 is arranged in parallel with the opposite side surface 503 of the particle capturing unit 501 at a predetermined distance. The first opposite side space 572 is in contact with the particle capturing unit 501, and the second opposite side space 573 is not in contact with the particle capturing unit 501.

The pore sealing sheet 571 is deformable so as to be adherable to the opposite side surface 503 of the particle capturing unit 501 in a case of sealing the pore. That is, the pore sealing sheet 571 is deformable from a state of being arranged in parallel with the opposite side surface 503 at a predetermined distance as described above to a state of adhering to the opposite side surface 503 of the particle capturing unit 501. In this deformation, a connection position of the pore sealing sheet 571 to the inner wall of the chamber 500 does not have to change. Since the pore sealing sheet 571 is deformable in this manner, a distance between the pore and the pore sealing sheet 571 may be adjusted. The pore sealing sheet 571 comes into contact with the opposite side surface 503 by the adjustment of the distance, so that the pore is sealed with the pore sealing sheet 571.

The pore sealing sheet 571 may be formed by using a material that enables such deformation. The material may be appropriately selected by those skilled in the art, and examples thereof may include polyvinylidene chloride, polyethylene, and polyvinyl chloride, for example.

The particle capturing chamber 500 is provided with a particle capturing channel unit 511, a first fluid supply channel unit 512, a second fluid supply channel unit 513, and a first fluid discharge channel unit 514. The particle capturing chamber 500 is further provided with third and fourth fluid supply channel units 520 and 521, and second and third fluid discharge channel units 522 and 523. That is, a total of eight channel units are connected to the particle capturing chamber 500. A valve may be provided on each of the above-described eight channel units.

The fourth fluid supply channel unit 521 and the third fluid discharge channel unit 523 are connected to the second opposite side space 573.

The particle capturing channel unit 511 and the second fluid supply channel unit 513 are connected to the first opposite side space 572.

The first fluid supply channel unit 512 and the first fluid discharge channel unit 514 are connected to the first settling side space 552.

The third fluid supply channel unit 520 and the second fluid discharge channel unit 522 are connected to the second settling side space 553.

FIG. 5B illustrates a manufacturing example of the particle capturing chamber 500. As illustrated in FIG. 5B, the particle capturing chamber 500 may be formed by, for example, stacking a glass plate 1, silicon resin sheets 2 and 3, a particle capturing chip 4, silicon resin sheets 5 and 6, and an acrylic resin lid 7 in this order. Furthermore, prior to the stacking, the above-described six channel units are formed in the respective layers as illustrated in FIG. 5B. A method for forming the channel in each layer may be appropriately selected by those skilled in the art.

The glass plate 1, the silicon resin sheets 2 and 3, and the particle capturing chip 4 form the settling side space 509 in FIG. 5A.

The particle capturing chip 4, the silicon resin sheets 5 and 6, and the acrylic resin lid 7 define the opposite side space 510 in FIG. 5A.

A well sealing sheet 8 is arranged between the silicon resin sheets 2 and 3. The well sealing sheet 8 is arranged so as to divide the settling side space into upper and lower two spaces. The upper space corresponds to the first settling side space 552 in FIG. 5A, and the lower space corresponds to the second settling side space 553 in FIG. 5A.

A well sealing sheet 9 is arranged between the silicon resin sheets 5 and 6. The well sealing sheet 9 is arranged so as to divide a space opposite to the settling side space into upper and lower two spaces. The upper space corresponds to the second opposite side space 573 in FIG. 5A, and the lower space corresponds to the first opposite side space 572 in FIG. 5A.

(3-2) Example of Operation Procedure

An example of a particle obtaining method according to the present technology using the particle capturing chamber 500 illustrated in FIG. 5A is hereinafter described.

(Step a)

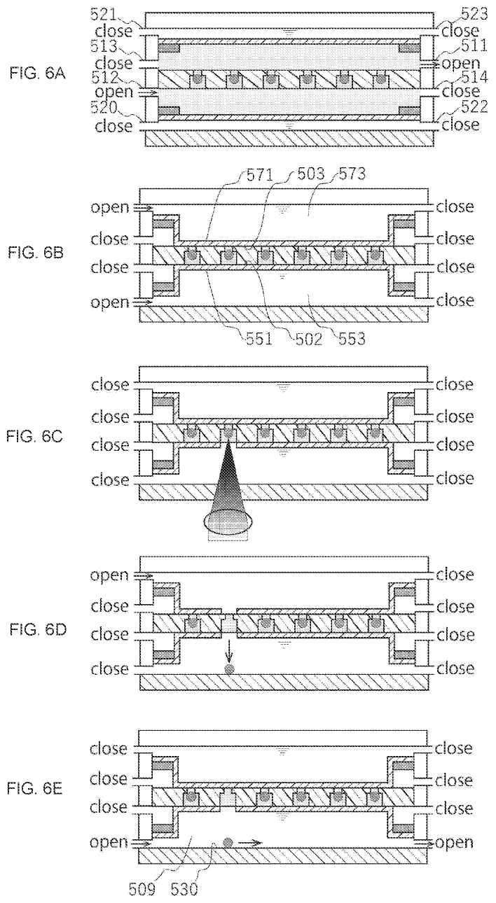

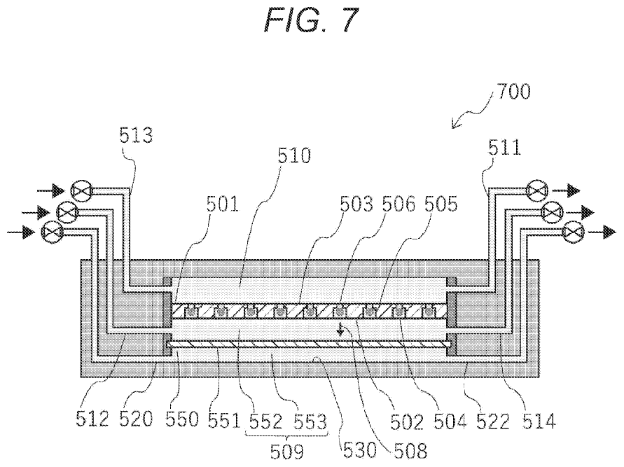

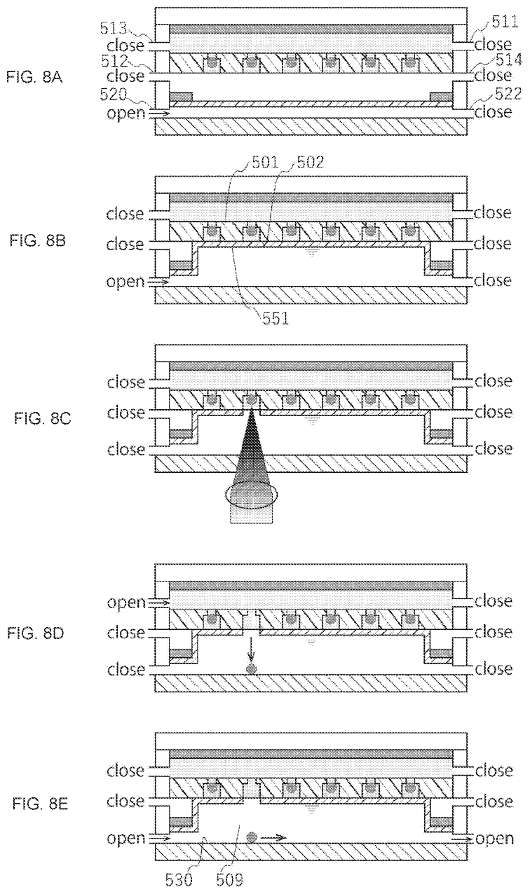

A cell is captured in the well as described in “(1-2-1) Particle capturing step” described above. As a result of the cell capturing, one cell is captured in each well as illustrated in FIG. 6(a).

For example, the valves on the first fluid supply channel unit 512 and the particle capturing channel unit 511 are opened and the other valves are closed, then cell-containing liquid is introduced from the first fluid supply channel unit 512 to the first settling side space 552 and suction is performed through the particle capturing channel unit 511. Therefore, the cell is captured in the well.

(Step b)

By introducing the liquid from the third fluid supply channel unit 520 into the second settling side space 553, the well sealing sheet 551 is deformed and adheres to the particle capturing surface 502 of the particle capturing unit 501. The well sealing sheet 551 comes into contact with the particle capturing surface 502, so that the well is sealed with the well sealing sheet 551. For the sealing, for example, the valve on the third fluid supply channel unit 520 may be opened, and the valves on the first fluid supply channel unit 512, the first fluid discharge channel unit 514, and the second fluid discharge channel unit 522 may be closed.

By introducing the liquid from the fourth fluid supply channel unit 521 into the second opposite side space 573, the pore sealing sheet 571 is deformed and adheres to the opposite side surface 503 of the particle capturing unit 501. The pore sealing sheet 571 comes into contact with the opposite side surface 503, so that the pore is sealed with the pore sealing sheet 571. For the sealing, for example, the valve on the fourth fluid supply channel unit 521 may be opened, and the valves on the particle capturing channel unit 511, the second fluid supply channel unit 513, and the third fluid discharge channel unit 523 may be closed.

At step b, as illustrated in FIG. 6(b), the sheets are adhered to both the particle capturing surface 502 and the opposite side surface 503 of the particle capturing unit 501. Therefore, the cell in the well is isolated in the space closed by a well wall, a pore wall, the well sealing sheet, and the pore sealing sheet. By isolating the cell in such closed space, for example, oxygen consumption of the cell may be analyzed.

(Step c)

The cell in the well is observed. The observation may be performed, for example, by an inverted microscope arranged below the particle capturing chamber 500 as illustrated in FIG. 6(c). As a result of the observation, a cell that should be obtained is selected.

During the observation, all the valves on the above-described eight channel units may be closed. This may prevent occurrence of a flow in the chamber and the observation of the cell becomes easier.

(Step d)

A portion corresponding to the well in which the cell selected at step c is captured of the well sealing sheet 551 is burned out by a laser beam. Therefore, a hole is made in the sheet portion that seals the selected well as illustrated in FIG. 6D. In a case where the well sealing sheet 551 contains an infrared light absorber, an infrared laser beam may be used as the laser beam.

During the hole making, all the valves on the above-described eight channel units may be closed. This may prevent occurrence of a flow in the chamber and the hole may be made more accurately.

The cell in the well freely falls through the hole on the bottom surface 530 of the settling side space 509.

Alternatively, the portion corresponding to the well of not only the well sealing sheet 551 but also the pore sealing sheet 571 may be burned out by the laser beam. Therefore, for example, as illustrated in FIG. 6(d), a hole may be formed in the portion corresponding to the well in which the selected cell is captured in both the well sealing sheet 551 and the pore sealing sheet 571. Thereafter, by pressurizing from the fourth fluid supply channel unit 521, it is possible to promote the fall of the particle.

(Step e)

As illustrated in FIG. 6(e), the cell that falls on the bottom surface 530 of the settling side space 509 may be recovered outside the particle capturing chamber 500 through the second fluid discharge channel unit 522.

During the recovery, for example, the valve on the third fluid supply channel unit 520 and the valve on the second fluid discharge channel unit 522 are opened, and the other valves are closed. Then, by suction by a pump (not illustrated) connected to the second fluid discharge channel unit 522 and pressurization by a pump connected to the third fluid supply channel unit 520, the cell is recovered through the second fluid discharge channel unit 522.

At steps a to e described above, it is possible to selectively recover only a desired cell. Furthermore, a plurality of desired cells may be selectively recovered by repeating steps d and e. Furthermore, by making a plurality of holes at step d, a plurality of selected particles may be collectively recovered at step e.

(3-3) Example of Particle Capturing Chamber

A particle capturing chamber obtained by eliminating the pore sealing unit 570 from the particle capturing chambers 500 illustrated in FIG. 5 may be used in the particle obtaining method of the present technology. An example of such particle capturing chamber is illustrated in FIG. 7.

A particle capturing chamber 700 illustrated in FIG. 7 is provided with a particle capturing unit 501. The particle capturing unit 501 is the same as the particle capturing unit 501 described in “(3-1) Example of particle capturing chamber” described above.

The settling side space 509 is provided with a sealing unit 550. The sealing unit 550 is the same as the sealing unit 550 described in “(3-1) Example of particle capturing chamber” described above. That is, the settling side space 509 is divided into a first settling side space 552 and a second settling side space 553 by a well sealing sheet 551 of the sealing unit 550. The well sealing sheet 551 is arranged in parallel with the particle capturing surface 502 of the particle capturing unit 501 at a predetermined distance. The first settling side space 552 is in contact with the particle capturing unit 501, and the second settling side space 553 is not in contact with the particle capturing unit 501.

Unlike the particle capturing chamber 500 described in “(3-1) Example of particle capturing chamber” described above, an opposite side space 510 is not provided with a pore sealing unit 570.

The particle capturing chamber 700 is provided with a particle capturing channel unit 511, a first fluid supply channel unit 512, a second fluid supply channel unit 513, and a first fluid discharge channel unit 514. The particle capturing chamber 700 is further provided with a third fluid supply channel unit 520 and a second fluid discharge channel unit 522.

Unlike the particle capturing chamber 500 described in “(3-1) Example of particle capturing chamber” described above, the particle capturing chamber 700 does not include a fourth fluid supply channel unit 521 and a third fluid discharge channel unit 523.

As described above, a total of six channel units are connected to the particle capturing chamber 700. A valve (not illustrated) is provided on each of the above-described six channel units.

The particle capturing channel unit 511 and the second fluid supply channel unit 513 are connected to the opposite side space 510.

The first fluid supply channel unit 512 and the first fluid discharge channel unit 514 are connected to the first settling side space 552.

The third fluid supply channel unit 520 and the second fluid discharge channel unit 522 are connected to the second settling side space 553.

(3-4) Example of Operation Procedure

An example of a particle obtaining method according to the present technology using the particle capturing chamber 700 illustrated in FIG. 7 is hereinafter described.

(Step a)

A cell is captured in the well as described in “(1-2) Particle capturing step” described above. As a result of the cell capturing, one cell is captured in each well as illustrated in FIG. 8(a).

For example, the valves on the first fluid supply channel unit 512 and the particle capturing channel unit 511 are opened and the other valves are closed, then cell-containing liquid is introduced from the first fluid supply channel unit 512 to the first settling side space 552 and suction is performed through the particle capturing channel unit 511. Therefore, the cell is captured in the well.

(Step b)

By introducing the liquid from the third fluid supply channel unit 520 into the second settling side space, the well sealing sheet 551 is deformed and adheres to the particle capturing surface 502 of the particle capturing unit 501. The well sealing sheet 551 comes into contact with the particle capturing surface 502, so that the well is sealed with the well sealing sheet 551. For this sealing, for example, the valve on the third fluid supply channel unit 520 may be opened and all other valves may be closed.

At step b, as illustrated in FIG. 8(b), the sheet adheres to the particle capturing surface 502 of the particle capturing unit 501.

(Step c)

The cell in the well is observed. The observation may be performed, for example, by an inverted microscope arranged below the particle capturing chamber 700 as illustrated in FIG. 8(c). As a result of the observation, a cell that should be obtained is selected.

During the observation, all the valves on the above-described six channel units may be closed. This may prevent occurrence of a flow in the chamber and the observation of the cell becomes easier.

(Step d)

A portion corresponding to the well in which the cell selected at step c is captured of the well sealing sheet 551 is burned out by a laser beam. Therefore, a hole is made in the sheet portion that seals the selected well as illustrated in FIG. 8(d). In a case where the well sealing sheet 551 contains an infrared light absorber, an infrared laser beam may be used as the laser beam. The cell in the well freely falls through the hole on the bottom surface 530 of the settling side space 509. Alternatively, it is possible to pressurize from the opposite side space 510. This may urge the cell to fall on the bottom surface 530 of the chamber.

During the hole making also, all the valves on the above-described six channel units may be closed. This may prevent occurrence of a flow in the chamber and the hole may be made more accurately.

(Step e)

As illustrated in FIG. 8(e), the cell that falls on the bottom surface 530 of the settling side space 509 may be recovered outside the particle capturing chamber 700 through the second fluid discharge channel unit 522.

During the recovery, for example, the valve on the third fluid supply channel unit 520 and the valve on the second fluid discharge channel unit 522 are opened, and the other valves are closed. Then, by suction by a pump (not illustrated) connected to the second fluid discharge channel unit 522 and pressurization by a pump connected to the third fluid supply channel unit 520, the cell is recovered through the second fluid discharge channel unit 522.

At steps a to e described above, it is possible to selectively recover only a desired cell. Furthermore, a plurality of desired cells may be selectively recovered by repeating steps d and e. Furthermore, by making a plurality of holes at step d, a plurality of selected particles may be collectively recovered at step e.

### (4) Third Example of First Embodiment (Particle Obtaining Method)

According to still another aspect of the present technology, the particle capturing unit may contain an elastic material, and the well may move toward the sheet by a pressure generated by injection of fluid and a distance between the well and the sheet may be adjusted.

An example of a particle capturing chamber in this aspect is hereinafter described, and an example of a particle obtaining method using the particle capturing chamber is next described.

(4-1) Example of Particle Capturing Chamber

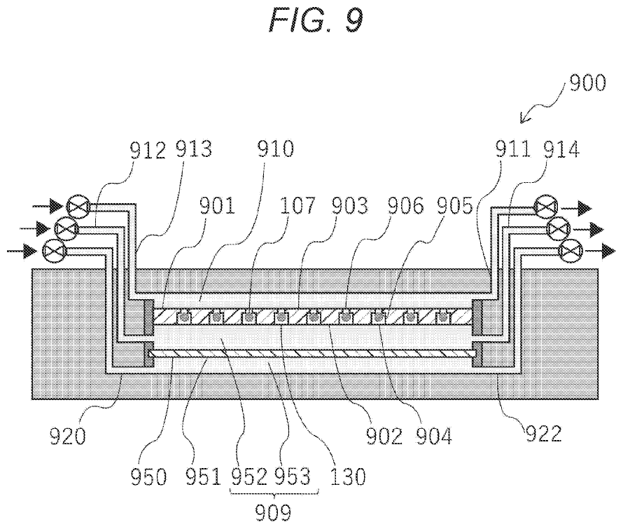

FIG. 9 illustrates an example of a particle capturing chamber of the present technology. A particle capturing chamber 900 illustrated in FIG. 9 is provided with a particle capturing unit 901 as the particle capturing chamber 100 illustrated in FIG. 1B described above. As the particle capturing unit 101 in FIG. 1B, the particle capturing unit 901 includes a particle capturing surface 902, a surface 903 on an opposite side, a plurality of wells 904 on the particle capturing surface 902, and a pore 906 penetrating from a bottom 905 of the well 904 to the opposite side surface 903. The particle capturing unit 901 divides the interior of the chamber 900 into a particle settling side space 909 and a space 910 on an opposite side.

The particle capturing unit 901 contains an elastic material. It is configured such that the particle capturing surface 902 may come into contact with a well sealing sheet 951 of a sealing unit 950 when a portion formed by using the elastic material bends. An entire particle capturing unit 901 may be formed by using the elastic material, or only a bending portion may be formed by using the elastic material. The elastic material is, for example, a silicone resin, more preferably PDMS. Since the particle capturing unit 901 bends, a distance between the well 904 and the well sealing sheet 951 is adjusted. The particle capturing surface 902 comes into contact with the well sealing sheet 951 by the adjustment of the distance, so that the well is sealed with the well sealing sheet 951.

The particle capturing chamber 900 is provided with the sealing unit 950 including the well sealing sheet 951. The sealing unit 950 is provided so as to divide the settling side space 909 into upper and lower two spaces (a first settling side space 952 and a second settling side space 953). The well sealing sheet 951 of the sealing unit 950 has rigidity sufficient for ensuring sealing of the well 904 when the particle capturing unit 901 comes into contact with the same. For example, the well sealing sheet 951 may be, for example, a glass sheet or a sheet having the same level of rigidity as the glass sheet. A thickness of the well sealing sheet 951 may be, for example, 20 μm or more, especially 30 μm to 100 μm, and more especially 40 μm to 60 μm. The glass sheet may have a thickness appropriately selected by those skilled in the art so as not to be damaged even if this comes into contact with the particle capturing unit 901. The glass sheet may contain the near-infrared light absorber described above.

The particle capturing chamber 900 is provided with a particle capturing channel unit 911, a first fluid supply channel unit 912, a second fluid supply channel unit 913, and a first fluid discharge channel unit 914. The particle capturing chamber 900 is further provided with a third fluid supply channel unit 920 and a second fluid discharge channel unit 922.

As described above, a total of six channel units are connected to the particle capturing chamber 900. A valve (not illustrated) is provided on each of the above-described six channel units.

The particle capturing channel unit 911 and the second fluid supply channel unit 913 are connected to the opposite side space 910.

The first fluid supply channel unit 912 and the first fluid discharge channel unit 914 are connected to the first settling side space 952.

The third fluid supply channel unit 920 and the second fluid discharge channel unit 922 are connected to the second settling side space 953.

(4-2) Example of Operation Procedure

(Step a)

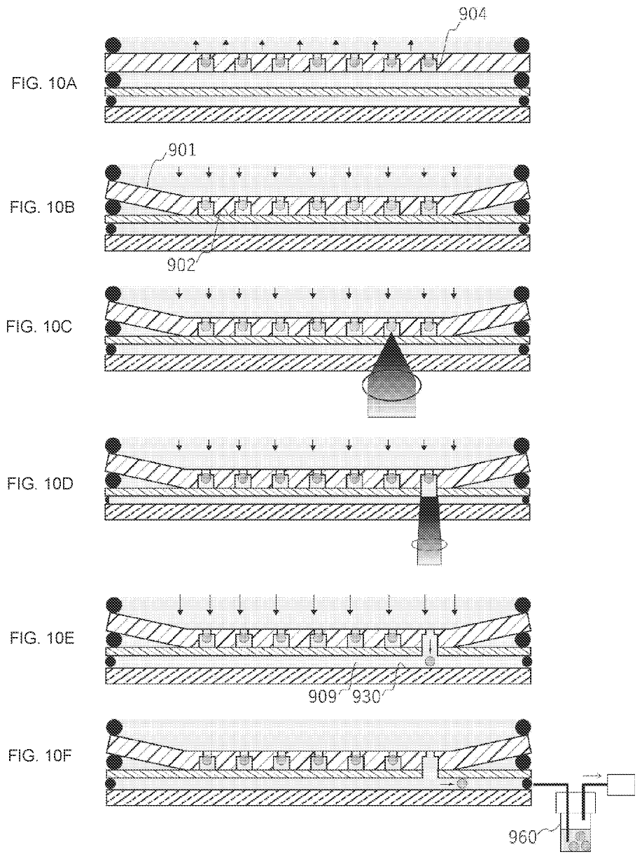

A cell is captured in the well as described in “(1-2-1) Particle capturing step” described above. As a result of the cell capturing, one cell is captured in each well 904 as illustrated in FIG. 10(a).

For example, the valves on the first fluid supply channel unit 912 and the particle capturing channel unit 911 are opened and the other valves are closed, then cell-containing liquid is introduced from the first fluid supply channel unit 912 to the first settling side space 952 and suction is performed through the particle capturing channel unit 911. Therefore, the cell is captured in the well. The suction may be performed at a suction pressure of, for example, 0.1 kPa.

(Step b)

A pressure is applied from the second fluid supply channel unit 913 to the opposite side space 910. Therefore, the particle capturing unit 901 is deformed. Along with the deformation, the well 904 moves toward the well sealing sheet 951. The particle capturing surface 902 comes into contact with the well sealing sheet 951 by the movement, so that the well is sealed with the well sealing sheet 951. For the sealing, the pressure is applied from the second fluid supply channel unit 913, for example, with the valve on the second fluid supply channel unit 913 opened and the other valves closed.

At step b, as illustrated in FIG. 10(b), the sheet adheres to the particle capturing surface 902 of the particle capturing unit 901.

The pressure applied at step b may be appropriately set by those skilled in the art depending on, for example, a size and a material of the particle capturing unit. For example, when a circular sheet having a diameter of 18 mm formed by using silicone resin MS1001 (Dow Corning Toray Co., Ltd.) as a material of the particle capturing unit was pressurized at 1 kPa, the central portion moved by 0.36 mm in a thickness direction. Therefore, in a case where the particle capturing unit 901 is the circular sheet having the diameter of 18 mm formed by using MS1001, by setting a distance between the particle capturing unit 901 and the well sealing sheet 951 to, for example, 0.2 mm to 0.3 mm and by applying a pressure of about 1 kPa, it is possible to seal the well 904 with the well sealing sheet 951. In this manner, the pressure may be set in consideration of the material of the particle capturing unit and ease of deformation of the particle capturing unit.

Furthermore, by adjusting the distance, for example, by setting the same to 0.2 mm to 0.3 mm as described above, channel resistance until the cell reaches the well may be reduced, so that it is possible to decrease a differential pressure required for capturing the cell in the well. Furthermore, it is also possible to prevent capturing of an unnecessary particle in the well.

Furthermore, depending on combination of the type of the material forming the well and the type of the cell, a force such as an intermolecular force and an electrostatic force acts between the well and the cell. Therefore, there is a case where a relatively large force is required to expel the cell from the well. For example, there is a case where a pressure required to deform the particle capturing unit 901 as described above is smaller than a pressure required to expel the cell from the well (for example, 2 kPa to 3 kPa). In this case, the cell is not expelled from the well even when the pressure is applied to deform the particle capturing unit 901 as described above.

(Step c)

The cell in the well is observed. The observation may be performed, for example, by an inverted microscope arranged below the particle capturing chamber 900 as illustrated in FIG. 10(c). As a result of the observation, a cell that should be obtained is selected.

During the observation, all the valves on the above-described six channel units may be closed. This may prevent occurrence of a flow in the chamber and the observation of the cell becomes easier.

Furthermore, since the well 904 of the particle capturing unit 901 moves to the well sealing sheet 951, a distance from a lens of the microscope to the cell is reduced, and high-definition observation of the cell using a high numerical aperture (NA) lens such as a liquid immersion lens or an oil immersion lens, for example, becomes easier. For example, if the distance between the particle capturing unit 901 and the well sealing sheet 951 is 0.2 mm to 0.3 mm as described above, a working distance (WD) is narrowed by that distance. Especially the number of choices of the high NA lens with WD 0.3 mm increases.

Furthermore, for example, in a case where the particle capturing unit 901 is formed by using a silicone resin, the particle capturing unit 901 is easily deformed, and winding occurs when the particle capturing unit 901 comes into contact with the well sealing sheet 951. Since the well sealing sheet 951 is formed by using the glass sheet or the sheet having the same level of rigidity as the glass sheet, the occurrence of the winding may be prevented.

(Step d)

A portion corresponding to the well in which the cell selected at step c is captured of the well sealing sheet 951 is burned out by a laser beam. Therefore, a hole is made in the sheet portion that seals the selected well as illustrated in FIG. 10(d). In a case where the well sealing sheet 951 contains an infrared light absorber, an infrared laser beam may be used as the laser beam.

During the hole making also, all the valves on the above-described six channel units may be closed. This may prevent occurrence of a flow in the chamber and the hole may be made more accurately.

After the hole making, the valve on the particle capturing channel unit 911 and/or the second fluid supply channel unit 913 is opened, then liquid may be introduced from the particle capturing channel unit 911 and/or the second fluid supply channel unit 913 to the opposite side space 972 of the chamber 900. This may urge the cell to fall on the bottom surface 930 of the chamber. The pressure for introducing the liquid may be larger than the pressure applied at step b, for example, 2 to 3 kPa as described above.

(Step e)

Through the hole made at step d, the cell falls on the bottom surface 930 of the settling side space 909 as illustrated in FIG. 10(e). Then, by suction through the second fluid discharge channel unit 922, the cell moves toward the second fluid discharge channel unit 922 in the settling side space 909 as illustrated in FIG. 10(f). Then, the cell may be recovered in a container 960 connected to the outside of the particle capturing chamber 900.

During the recovery, for example, the valve on the third fluid supply channel unit 920 and the valve on the second fluid discharge channel unit 922 are opened, and the other valves are closed. Then, by suction by a pump (not illustrated) connected to the second fluid discharge channel unit 922 and pressurization by a pump connected to the third fluid supply channel unit 920, the cell is recovered in the container 960 through the second fluid discharge channel unit 922.

At steps a to e described above, it is possible to selectively recover only a desired cell. Furthermore, a plurality of desired cells may be selectively recovered by repeating steps d and e. Furthermore, by making a plurality of holes at step d, a plurality of selected particles may be collectively recovered at step e.

### 2. Second Embodiment (Particle Capturing Chamber)

The present technology provides a particle capturing chamber provided with a particle capturing unit including at least one well for capturing a particle therein, and a sealing unit including a sheet for sealing the well, in which a distance between the well and the sheet is adjustable. It is possible to execute the particle obtaining method as described in “1. First embodiment (particle obtaining method)” described above by the particle capturing chamber. Therefore, a desired particle may be selectively recovered.

The particle capturing chamber of the present technology is as described in “1. First embodiment (particle obtaining method)” described above. More specifically, the particle capturing chamber of the present technology is as described in “(1-1) Particle capturing chamber”, “(2-1) Example of particle capturing chamber”, “(3-1) Example of particle capturing chamber”, “(3-3) Example of particle capturing chamber”, and “(4-1) Example of particle capturing chamber” in “1. First embodiment (particle obtaining method) described above These descriptions apply to the particle capturing chamber of the present technology.

### 3. Third Embodiment (Particle Analysis System)

The present technology provides a particle analysis system provided with a particle capturing chamber provided with a particle capturing unit including at least one well for capturing a particle therein and a sealing unit including a sheet for sealing the well, an analysis unit that analyzes the captured particle, and a light source that perforates the sheet on the basis of information of the particle analyzed by the analysis unit.

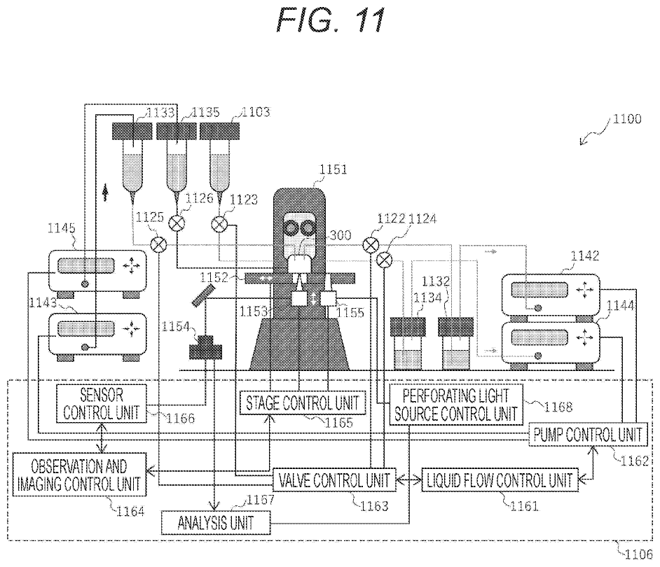

An example of the particle analysis system of the present technology is described with reference to FIG. 11. FIG. 11 is a view illustrating a configuration example of the particle analysis system of the present technology.

A particle analysis system 1100 of the present technology illustrated in FIG. 11 is provided with the particle capturing chamber 300 described in “(2-1) Example of particle capturing chamber” in 1. described above. Note that any of the other particle capturing chambers described in 1. described above may be adopted in place of the particle capturing chamber 300.

Out of components of the particle capturing chamber 300, to the first fluid supply channel unit 312, a liquid supply tank 1103 as a fluid supply unit is connected through a valve 1123.

Furthermore, to the second fluid supply channel unit 313, a liquid supply tank 1133 is connected through a valve 1125. A micropressure pump 1143 is connected to the liquid supply tank 1133. It is possible to supply fluid into the particle capturing chamber 300 by driving the micropressure pump 1143.

To the particle capturing channel unit 311, a waste liquid tank 1132 and a micropressure pump 1142 are connected through a valve 1122.

To the fluid discharge channel unit 314, a waste liquid tank 1134 and a micropressure pump 1144 are connected through a valve 1124. The waste liquid tank 1134 may be replaced with a particle recovery tank, for example, for particle recovery.

To the peeling fluid supply channel unit 360, a liquid supply tank 1135 and a micropressure pump 1145 are connected through a valve 1126.

The particle capturing chamber 300 is arranged on a stage 1152 of an inverted microscope 1151. The stage 1152 may be moved by electrical control, for example in X and Y directions.

An objective lens 1153 of the inverted microscope 1151 may be moved by electrical control, for example, in a Z direction. The objective lens 1153 is configured to be able to observe the particle capturing surface of the particle capturing chamber 300 from below the particle capturing chamber 300. The inverted microscope 1151 may further be provided with a light source for particle observation (for example, a halogen lamp, a mercury lamp, an LED or the like), a filter (for example, an excitation filter and/or a fluorescent filter), an objective lens having a magnification according to a purpose, an electric XY stage, and an electric Z stage (a stage that moves the objective lens or a stage on which the chamber is placed is applicable).

A camera 1154 is connected to the inverted microscope 1151. The camera 1154 is configured to be able to image the particle capturing surface of the particle capturing chamber 300 through the objective lens 1153. The camera 1154 includes, for example, a CMOS or CCD image sensor. The camera 1154 is configured to be able to transmit imaging data to an imaging data processing unit described below. The camera 1154 may be the camera capable of capturing a moving image, for example, for recording or observing a change in particle over time.

Furthermore, the particle analysis system 1100 also includes a light source 1155 that applies light for perforating a selected portion of the well sealing sheet 351. The light may be, for example, infrared light, especially near-infrared light.

The particle analysis system 1100 is provided with a control unit 1106. The control unit 1106 includes a liquid flow control unit 1161, a pump control unit 1162, a valve control unit 1163, an observation and imaging control unit 1164, a stage control unit 1165, a sensor control unit 1166, an analysis unit 1167, and a perforating light source control unit 1168.

The liquid flow control unit 1161 controls the pump control unit 1162 and the valve control unit 1163 to control the supply of the fluid into the particle capturing chamber 300 or the discharge of the fluid from the particle capturing chamber 300. The liquid flow control unit 1161 controls, for example, cell capturing, drug solution exchange, and/or cell recovery.

The pump control unit 1162 controls operation of the micropressure pump and/or a differential pressure applied by the micropressure pump.

The valve control unit 1163 controls opening and closing of the valve.

The observation and imaging control unit 1164 controls the stage control unit 1165 and the sensor control unit 1166 to image the particle capturing surface.

The stage control unit 1165 controls the stage 1152 and/or the objective lens 1153. The stage control unit 1165 may move an area to be imaged and/or adjust a focus. Furthermore, the stage control unit 1165 controls a position of the stage 1152 and/or the light source 1155. The control makes it possible to irradiate a sheet portion covering a well in which a desired particle is captured with light for perforating the portion.

The sensor control unit 1166 controls the camera 1154. The sensor control unit 1166 may control, for example, a timing of imaging the particle capturing surface, an exposure period, and/or the number of times of imaging.

The observation and imaging control unit 1164 may synchronize the stage control by the stage control unit 1165 and the camera operation control by the sensor control unit 1166. Furthermore, the observation and imaging control unit 1164 may control rotation of an electric revolver to which a plurality of objective lenses 1153 is attached. That is, the observation and imaging control unit 1164 may switch the objective lens 1153.

The analysis unit 1167 processes the imaging data transmitted from the camera 1154. For example, the analysis unit 1167 may analyze the particle on the basis of the imaging data. For example, the analysis unit 1167 may extract a shape of the particle and/or analyze fluorescence intensity on the basis of the imaging data. The data obtained as a result of the analysis may be presented to a user through an output device such as a display, for example. As a result, it is possible to assist the user in analyzing and/or diagnosing the particle. The user may select the particle that should be obtained on the basis of the analysis result.

The perforating light source control unit 1168 controls the light source 1155 to irradiate the portion that seals the well containing the selected particle with light for perforation, for example, near-infrared light. The irradiation makes a hole on the sheet that seals the well containing the selected particle. In order to irradiate a desired position with light, the perforating light source control unit 1168 may change the position of the light source 1155 or drive the stage control unit 1165 to change the position of the stage 1152.

Regarding the present technology described above, those skilled in the art understand that various changes, combinations, sub-combinations, or alternatives within the scope of the present technology and its equivalents are possible, for example, depending on design requirements or other factors.

Note that the present technology may also have a following configuration.

[1] A particle obtaining method provided with:

a particle capturing step of capturing a particle in a well;

a sealing step of sealing the well with a sheet; and

a particle obtaining step of obtaining the particle from a selected well after the sealing step.

[2] The particle obtaining method according to [1],

in which, at the particle capturing step, the particle is captured in the well by suction to a side opposite to a settling side of the particle through a pore provided in the well.

[3] The particle obtaining method according to [1] or [2],

in which, at the sealing step, the well is sealed with the sheet by adjustment of a distance between the well and the sheet.

[4] The particle obtaining method according to any one of [1] to [3], further provided with:

a selecting step of observing the particle in the well and selecting the particle that should be obtained after the sealing step.

[5] The particle obtaining method according to any one of [1] to [4], further provided with:

a hole making step of making a hole in a portion that seals the selected well of the sheet after the sealing step,

in which the particle is obtained from the hole made at the hole making step at the particle obtaining step.

[6] A particle capturing chamber provided with:

a particle capturing unit that includes at least one well for capturing a particle in the well; and

a sealing unit including a sheet for sealing the well,

in which a distance between the well and the seat is adjustable.

[7] The particle capturing chamber according to [6],

in which the distance between the well and the sheet is adjusted by movement of the sheet toward the well or movement of the well toward the sheet, and

the well is sealed with the sheet by the movement.

[8] The particle capturing chamber according to [6] or [7],

in which the sheet is transparent.

[9] The particle capturing chamber according to any one of [6] to [8],

in which the sheet further includes an adhesive layer.

[10] The particle capturing chamber according to any one of [6] to [9],

in which the sheet includes a piezoelectric body and the piezoelectric body emits an elastic wave.

[11] The particle capturing chamber according to any one of [6] to [10],

in which the sheet further includes a reagent layer.

[12] The particle capturing chamber according to any one of [6] to [11],

in which the sealing unit further includes a support layer stacked on the sheet, the sheet is peeled from the support layer by a pressure generated by injection of fluid, and the distance between the well and the sheet is adjusted.

[13] The particle capturing chamber according to [12],

in which the fluid is at least one selected from water, air, oil, and a cell culture solution.

[14] The particle capturing chamber according to any one of [6] to [11],

in which a pore used for capturing the particle in the well by suction is provided on each of the at least one well,

the particle capturing chamber further provided with:

a pore sealing unit including a sheet for sealing the pore.

[15] The particle capturing chamber according to any one of [6] to [11],

in which the particle capturing unit includes an elastic material, the well is moved toward the sheet by a pressure generated by injection of fluid, and the distance between the well and the sheet is adjusted.

[16] The particle capturing chamber according to any one of [6] to [15],

in which a pore used for capturing the particle in each well by suction is provided on each of the at least one well, and the well opens toward a settling side of the particle.

[17] A particle analysis system provided with:

a particle capturing chamber provided with a particle capturing unit including at least one well for capturing a particle in the well and a sealing unit including a sheet for sealing the well;

an analysis unit that analyzes the captured particle; and

a light source that perforates the sheet on the basis of information of the particle analyzed by the analysis unit.

### REFERENCE SIGNS LIST

100A, 100B Particle capturing chamber
101 Particle capturing unit
102 Particle capturing surface
103 Surface on side opposite to particle capturing surface
104 Well
105 Well bottom
106 Pore

1. A particle obtaining method comprising:
a particle capturing step of capturing a particle in a well;
a sealing step of sealing the well with a sheet; and
a particle obtaining step of obtaining the particle from a selected well after the sealing step.

2. The particle obtaining method according to claim 1,
wherein, at the particle capturing step, the particle is captured in the well by suction to a side opposite to a settling side of the particle through a pore provided in the well.

3. The particle obtaining method according to claim 1,
wherein, at the sealing step, the well is sealed with the sheet by adjustment of a distance between the well and the sheet.

4. The particle obtaining method according to claim 1, further comprising:
a selecting step of observing the particle in the well and selecting the particle that should be obtained after the sealing step.

5. The particle obtaining method according to claim 1, further comprising:
a hole making step of making a hole in a portion that seals the selected well of the sheet after the sealing step,
wherein the particle is obtained from the hole made at the hole making step at the particle obtaining step.

6. A particle capturing chamber comprising:
a particle capturing unit that includes at least one well for capturing a particle in the well; and
a sealing unit including a sheet for sealing the well,
wherein a distance between the well and the seat is adjustable.

7. The particle capturing chamber according to claim 6,
wherein the distance between the well and the sheet is adjusted by movement of the sheet toward the well or movement of the well toward the sheet, and
the well is sealed with the sheet by the movement.

8. The particle capturing chamber according to claim 6,
wherein the sheet is transparent.

9. The particle capturing chamber according to claim 6,
wherein the sheet further includes an adhesive layer.

10. The particle capturing chamber according to claim 6,
wherein the sheet includes a piezoelectric body and the piezoelectric body emits an elastic wave.

11. The particle capturing chamber according to claim 6,
wherein the sheet further includes a reagent layer.

12. The particle capturing chamber according to claim 6,
wherein the sealing unit further includes a support layer stacked on the sheet, the sheet is peeled from the support layer by a pressure generated by injection of fluid, and the distance between the well and the sheet is adjusted.

13. The particle capturing chamber according to claim 12,
wherein the fluid is at least one selected from water, air, oil, and a cell culture solution.

14. The particle capturing chamber according to claim 6,
wherein a pore used for capturing the particle in the well by suction is provided on each of the at least one well,
the particle capturing chamber further comprising:
a pore sealing unit including a sheet for sealing the pore.

15. The particle capturing chamber according to claim 6,
wherein the particle capturing unit includes an elastic material, the well is moved toward the sheet by a pressure generated by injection of fluid, and the distance between the well and the sheet is adjusted.

16. The particle capturing chamber according to claim 6,
wherein a pore used for capturing the particle in each well by suction is provided on each of the at least one well, and the well opens toward a settling side of the particle.

17. A particle analysis system comprising:
a particle capturing chamber provided with a particle capturing unit including at least one well for capturing a particle in the well and a sealing unit including a sheet for sealing the well;
an analysis unit that analyzes the captured particle; and
a light source that perforates the sheet on a basis of information of the particle analyzed by the analysis unit.
